(19) United States
(12) Patent Application Publication
(10) Pub. No.: US 2012/0123400 A1
(43) Pub. Date: May 17, 2012

(54) METHODS AND DEVICES FOR CONTROLLING ENERGY DURING ABLATION

(76) Inventors: David Francischelli, Anoka, MN (US); Catherine R. Condie, Shoreview, MN (US); Jinback Hong, Maple Grove, MN (US)

(21) Appl. No.: 13/104,483

(22) Filed: May 10, 2011

Related U.S. Application Data
(60) Provisional application No. 61/333,100, filed on May 10, 2010.

(51) Int. Cl. A61B 18/12; A61B 18/18; A61B 18/02

(52) U.S. Cl. 606/20; 606/41; 606/33

(57) ABSTRACT

System and method for ablating tissue of a heart of a patient. The tissue is characterized, then a predetermined ablation procedure is selected based on the characterization, ablation energy is delivered according to procedure with the ablation device, and a temperature of the tissue and an impedance of the tissue are determined. Delivery of ablation energy is ceased at a time based, at least in part, on when at least one of an accumulated effective temperature of the tissue over time exceeds a thermal dose threshold and an accumulated effective energy of the tissue over time exceeds an effective energy threshold. Else, the ablation energy delivered is modified by adjusting the energy level based, at least in part, on at least one of the temperature being outside of a predetermined temperature range and the impedance being outside of an impedance range.

### CROSS-REFERENCE TO RELATED APPLICATIONS

This application claims priority under 35 U.S.C. §119(e)(1) to U.S. Provisional Patent Application Ser. No. 61/333,100, filed May 10, 2010, entitled “Methods and Devices for Controlling Energy During Ablation”, and bearing Attorney Docket No. P0033539.00; and the entire teachings of which are incorporated herein by reference.

### BACKGROUND

Atrial fibrillation is a common cardiac condition in which irregular heart beats cause a decrease in the efficiency of the heart, sometimes due to variances in the electrical conduction system of the heart. In some circumstances, atrial fibrillation poses no immediate threat to the health of the individual suffering from the condition but may, over time, result in conditions adverse to the health of the patient, including heart failure and stroke. But in the case of many of the individuals suffering from atrial fibrillation, symptoms affecting the patient's quality of life may occur immediately with the onset of the condition, including lack of energy, fainting and heart palpitations.

In some circumstances, atrial fibrillation may be treated with drugs or through the application of defibrillation shocks. In cases of persistent atrial fibrillation, however, surgery may be required. A surgical procedure originally developed to treat atrial fibrillation is known as a “MAZE” procedure, where the atria are surgically cut apart along specific lines and sutured back together. While possibly effective, the MAZE procedure tends to be complex and may require highly invasive access to the thorax. In order to reduce the need to open the atria, thermal ablation tools were developed to produce lines of inactive heart wall that mimic the MAZE procedure. This is most commonly done using radio frequency (RF) ablation devices to ablate and isolate tissue which may be responsible for the improper electrical conduction that causes atrial fibrillation. One such location of tissue which may be responsible for improper electrical conduction is at the junction of the pulmonary veins with the left atrium where spontaneous triggers for initiation of atrial fibrillation have been found. Patients who suffer from a paroxysmal form of atrial fibrillation experience short, self terminating episodes of atrial fibrillation. “Lone” atrial fibrillation occurs in patients who have either few or no other significant cardiac diseases.

While techniques have been developed to permit the relatively accurate and reliable placement of ablation members with respect to tissue which is desired to be ablated, the delivery of ablation has remained a relatively inexact process. In particular, while thermal damage may be required in order to create cellular necrosis to form a lesion in the tissue, excessive application of energy may result in excessive damage to tissue, such as charring of the tissue, damage which goes beyond desirable cellular necrosis. Such damage may further include perforation of the tissue, excessive surface damage, charring, and the bursting of pockets of heated gasses within the tissue, known as “popping”. The significance of such events may vary. Occurrences of perforation may create an actual risk of harm to a patient and may require remedial response to repair damage. Occurrences of popping may merely be startling and unnerving to the patient or physician. However, in each case, the effect may be undesirable, and the cause may be traced, at least in part, to unnecessarily and undesirably high rates of energy transfer to the tissue. In addition, particularly in the case of popping and perforation, it may be difficult or impossible to anticipate the event before it happens.

The delivery of too low of a rate of ablation energy may reduce the likelihood of such events occurring, but may carry with it other negative implications. In particular, if the rate of delivery is too minimal a lesion may not form at all, the lesion may be incomplete, or a lesion may form but over an excessively long a period of time to which the patient could be subjected. As such, ablation procedures typically ideally occur within a particular range which causes cellular necrosis at a rate neither too low nor too high.

However, the desirable range may not be consistent between and among patients and between and among various ablation locations within a single patient. Users have attempted to monitor real-time factors, such as a patient's electrogram. When the electrogram, for instance, decreases past a certain threshold during ablation the ablation energy may be dialed back in order to prevent excessive heating. This method, however, may not be highly accurate. In other cases, ultrasound imaging has been applied to tissue in order to detect heated gas bubbles or other changes within the tissue. Again, diagnosis in such circumstances may be unreliable and may be prone to subjective analysis.

### SUMMARY

Optimally, all ablation energy transmitted to tissue would contribute to the formation of a lesion. However, it may be the case that at least some energy is lost in ways not relating to lesion formation. It may be that three sources of energy loss may be conductive heat loss in adjacent tissue, conductive heat loss due to microcirculation and conductive heat loss due to intra-cardiac blood flow. These loss factors may contribute to a bioheat equation. However, the factors tend to vary from patient to patient. While one patient may have high conduction within the tissue, contributing to conductive heat loss in adjacent tissue, another patient may have relatively low conduction. Thus, the patient with low conduction may tend to be more prone to tissue damage during ablation if subjected to the same energy rate as the patient with high conduction. Similarly, a patient with relatively low blood flow may be more prone to tissue damage than a patient with relatively high blood flow.

By characterizing the tissue in and around the ablation zone before applying full ablation energy to the tissue, insight may be gained into the tissue and the delivery of ablation energy may be better dialed in before the delivery of full ablation energy. In particular, a test pulse may be delivered to the tissue and the response of tissue parameters measured. Based on the response of the tissue impedance and temperature to the test pulse, the tissue may be characterized.

In particular, because of the number of factors which contribute to the response of the tissue, the response of the impedance and temperature may be compared against one or more predetermined response curves. The predetermined response curves may be multi-order polynomials obtained and calibrated in prior clinical settings. The response curves may be determined and calibrated for different kinds of ablation devices.

In an embodiment, ablation energy is delivered, in one instance, to the tissue. A biological response to the ablation energy is sensed in the tissue. Then the biological response is compared with a plurality of predetermined mathematical models of predetermined biological responses of tissue to energy. One of a plurality of ablation procedures is selected based on a result from the comparing step. Ablation energy is delivered, in another instance, to the tissue in accordance with a selected one of the plurality of ablation procedures.

In an embodiment, the ablation energy creates a lesion in the tissue.

In an embodiment, the sensing a biological response step occurs after the delivering a first ablation pulse step.

In an embodiment, the delivering ablation energy, in one instance, step delivers a first pulse of ablation energy, and wherein the sensing a biological response step delivers a second pulse of ablation energy smaller than the first pulse.

In an embodiment, the second pulse of energy is less than an amount of energy necessary to ablate the tissue.

In an embodiment, the sensing a biological response step senses an impedance of the tissue.

In an embodiment, the sensing a biological response step occurs, at least in part, concurrently with the delivering ablation energy step.

In an embodiment, the biological response is a first biological response and further comprising the step, after the sensing a first biological response step, of sensing a second biological response in the tissue.

In an embodiment, the first biological response is an impedance of the tissue and the second biological response is a temperature of the tissue.

In an embodiment, the first biological response is a temperature of the tissue and the second biological response is an impedance of the tissue.

In an embodiment, the sensing a biological response is sensing an impedance of the tissue.

In an embodiment, the impedance is a complex impedance.

In an embodiment, the sensing a biological response senses a temperature of the tissue.

In an embodiment, the selecting step selects the ablation procedure from a plurality of predetermined ablation procedures.

In an embodiment, the plurality of ablation procedures is selected from a low power procedure, a long-term procedure, a high power procedure, a short-term procedure, a temperature set point procedure, a unipolar energy procedure, a bipolar energy procedure, a rise time procedure, cryo-energy procedure, a RF energy procedure, or any combination thereof.

In an embodiment, the ablation procedure is a series of ablation pulses delivered in sequence for a predetermined time.

In exemplary embodiments, the tissue ablated is any tissue of a subject that may benefit from ablation of the tissue, e.g., cardiac tissue, tumor tissue, etc. In one embodiment, the tissue includes heart tissue.

In an embodiment, the biological response is a function of a thickness of a wall of the heart.

In an embodiment, the second biological response is a function of flow of blood in the heart.

In an embodiment, each of the plurality of mathematical models is a polynomial mathematical model, or a logarithmic or other non-polynomial model.

In an embodiment, an ablation member is operatively coupled to the source of ablation energy and is adapted to provide ablation energy to the tissue. A sensing module senses a biological characteristic of the tissue to the ablation energy delivered to the tissue from the ablation member. A controller is operatively coupled to the source of energy and the sensing module. The controller controls the source of energy to deliver the ablation energy, for instance, to the tissue through the ablation member. The controller determines a biological response in the tissue based on the biological characteristic sensed by the sensing module. The controller further compares the biological response with a plurality of predetermined mathematical models of the biological response to energy to obtain a comparison. In addition, the controller selects an ablation procedure based on the comparison. The controller controls the source of energy to deliver the ablation energy, for instance, to the tissue through the ablation member based on a selected one of the plurality of ablation procedures.

In an embodiment, the controller creates a lesion in the tissue with the ablation energy delivered in one instance.

In an embodiment, the biological response occurs after delivery of the ablation energy.

In an embodiment, the ablation energy delivered in one instance is a first pulse and wherein the controller delivers a second pulse of energy smaller than the first pulse.

In an embodiment, the second pulse of energy is less than an amount of energy necessary to ablate the tissue.

In an embodiment, the biological response is an impedance of the tissue.

In an embodiment, the biological characteristic is sensed concurrently, at least in part, with delivery of the first pulse of ablation energy.

In an embodiment, the biological response is a first biological response and wherein the sensing module senses a second biological characteristic in the tissue and the controller determines a second biological response based on the second biological characteristic.

In an embodiment, the first biological response is an impedance of the tissue and the second biological response is a temperature of the tissue.

In an embodiment, the first biological response is a temperature of the tissue and the second biological response is an impedance of the tissue.

In an embodiment, the sensing a biological characteristic is sensing an impedance of the tissue.

In an embodiment, the biological response is a temperature of the tissue.

In an embodiment, the biological response is a first biological response and wherein the sensing module senses a second biological response in the tissue.

In an embodiment, a heart of a patient is ablated using an ablation device. Ablation energy is delivered at an energy level value to the tissue of the patient with the ablation device, and a value of a temperature of the tissue and a value of an impedance of the tissue at a plurality of measurement times are determined. Delivering ablation energy is ceased at a time based, at least in part, on when at least one of an accumulated effective temperature of the tissue over time exceeds a predetermined thermal dose threshold, the effective temperature occurring when the value of temperature exceeds a temperature value at which any cell necrosis of the tissue occurs, and an accumulated effective energy of the tissue over time exceeds a predetermined effective energy threshold, the effective energy occurring when the energy level exceeds a value of energy at which any cell necrosis occurs. If neither of the accumulated effective temperature exceeds the thermal dose threshold nor the accumulated effective energy exceeds the effective energy threshold, ablation delivery is modified by adjusting the energy level based, at least in part, on at least one of the temperature value being outside of a predetermined temperature range and the impedance value being outside of a predetermined impedance range and returning to the determining step.

In an embodiment, the delivering ablation energy step is ceased based, at least in part, on when both of the accumulated effective temperature of the tissue over time exceeds the predetermined thermal dose threshold, the effective temperature occurring when the value of temperature exceeds the temperature value at which any cell necrosis of the tissue occurs and the accumulated effective energy of the tissue over time exceeds the predetermined effective energy threshold, the effective energy occurring when the energy level exceeds the value of energy at which any cell necrosis occurs. If either of the accumulated effective temperature exceeds the thermal dose threshold nor the accumulated effective energy exceeds the effective energy threshold, modifying the delivering ablation energy step by adjusting the energy level based, at least in part, on at least one of the temperature value being outside of a predetermined temperature range and the impedance value being outside of an predetermined impedance range and returning to the determining step.

In an embodiment, the plurality of measurement times occur at intervals of less than one second.

In an embodiment, the intervals are one-fifth of a second.

In an embodiment, the accumulated effective temperature is based on the sum of temperature divided by a number of a plurality of measurement times which occur per second.

In an embodiment, the effective temperature is fifty-five degrees Celsius.

In an embodiment, the thermal dose threshold is 800-4800 degree-seconds, for example, 1000 degree-seconds.

In an embodiment, the accumulated effective energy is based on the energy level at each of the plurality of measurement times.

In an embodiment, the plurality of measurement times occur at intervals of less than one second.

In an embodiment, the intervals are one-fifth of a second.

In an embodiment, the ablation energy is delivered for a duration, and the delivering ablation energy step is ceased based, at least in part, on both of the accumulated effective temperature of the tissue over time exceeding the predetermined thermal dose threshold and the accumulated effective energy of the tissue over time exceeding the predetermined effective energy threshold, or the duration exceeding a duration threshold.

In an embodiment, the duration threshold is approximately one hundred twenty seconds.

In an embodiment, tissue of a heart of a patient is ablated using an ablation device. The tissue is characterized to obtain a characterization, which in one embodiment includes calculating the cease time. In certain embodiments, the characterization step includes determining the accumulated effective temperature, the thermal dose threshold, the effective energy, the effective energy threshold, or any combination thereof. One of a plurality of predetermined ablation procedures is selected based on the characterization, ablation energy is delivered according to the one of the plurality of ablation procedures at an energy level value to the tissue of the patient with the ablation device, a value of a temperature of the tissue and a value of an impedance of the tissue at a plurality of measurement times are determined. The delivering ablation energy step is ceased at a time based, at least in part, on when at least one of an accumulated effective temperature of the tissue over time exceeds a predetermined thermal dose threshold, the effective temperature occurring when the value of temperature exceeds a temperature value at which any cell necrosis of the tissue occurs and an accumulated effective energy of the tissue over time exceeds a predetermined effective energy threshold, the effective energy occurring when the energy level exceeds a value of energy at which any cell necrosis occurs. If neither of the accumulated effective temperature exceeds the thermal dose threshold nor the accumulated effective energy exceeds the effective energy threshold, the delivering ablation energy step is modified by adjusting the energy level based, at least in part, on at least one of the temperature value being outside of a predetermined temperature range and the impedance value being outside of a predetermined impedance range. Then the determining step is returned to.

### FIGURES

FIG. 1 is a cross-sectional illustration of the heart of a patient;

FIG. 2 is a combination isometric and block diagram of an ablation system for ablating the heart of the patient;

FIG. 3 is a graphical representation of a response of tissue of the heart of the patient to ablation energy;

FIG. 4 is a block diagram of a controller for controlling the delivery of ablation energy;

FIGS. 5A and 5B are graphs of predetermined response curves;

FIG. 6 is a flowchart for ablating tissue;

FIG. 7 is a flowchart for characterizing tissue before delivering ablation energy;

FIG. 8 is a flowchart for characterizing tissue according to an impedance measurement;

FIG. 9 is a flowchart for selecting an ablation power level according to impedance and temperature measurements; and

FIG. 10 is a combination isometric and block diagram of an ablation system having one impedance sensor and two thermocouples.

### DESCRIPTION

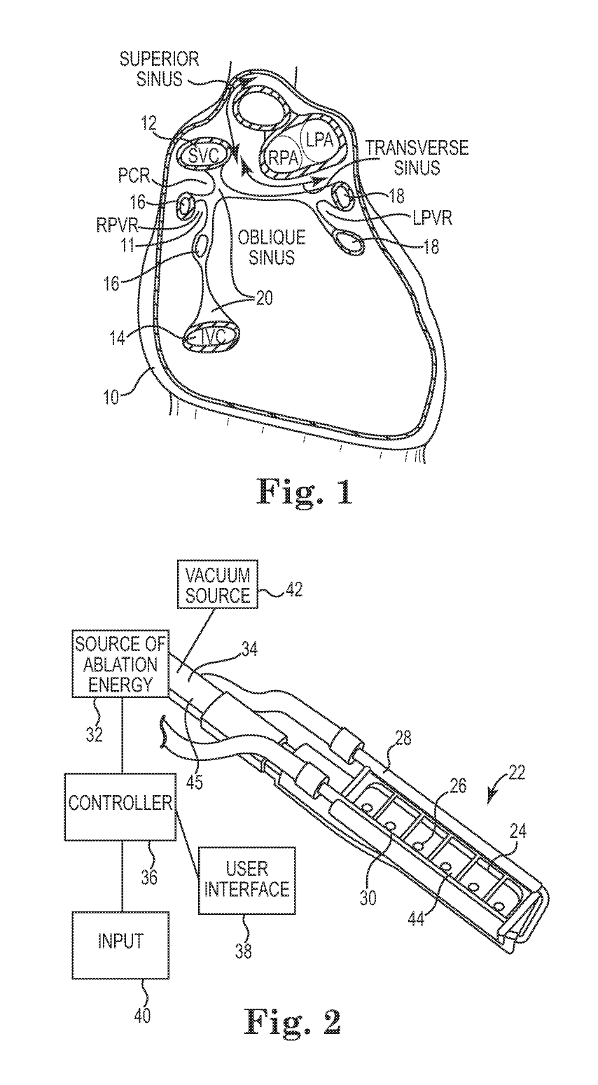

FIG. 1 shows a posterior view of a diagram of the great vessels extending posteriorly from the pericardial sac of the human heart 10, and the tissues 11 of heart 10. Superior vena cava 12 and inferior vena cava 14 deliver de-oxygenated blood to the heart from the upper and lower regions of the body, respectively. The two right pulmonary veins 16 and two left pulmonary veins 18, deliver oxygenated blood from the lungs to the left atrium. Pericardial reflections 20 extend between superior vena cava 12, inferior vena cava 14, right pulmonary veins 16 and left pulmonary veins 18.

FIG. 2 illustrates a combination isometric and block diagram of ablation system 22 for ablating tissue 11 of heart 10. Ablation system 22 includes head 24 which may incorporate multiple ablation members 26 and sensors 28, 30. In an embodiment ablation system 22 may include only one ablation member 26. In an embodiment, ablation system 22 may include only one sensor 28. In an embodiment, ablation member 26 is configured to deliver radio frequency energy. In various embodiments, ablation member 26 is configured to deliver ultrasound energy. In an embodiment, ablation member 26 is an electrode. In such an embodiment, ablation member 26 is configured to deliver ultrasound ablation energy in a manner well known in the art. Ablation member 26 is coupled to source of ablation energy 32 by way of a conductor disposed in neck 34.

Sensors 28, 30 are configured to sense at least one parameter in and around tissue 11 which is to be ablated. In an embodiment, sensor 28 is an impedance measuring sensor, such as an ohmmeter or an instrument which measures impedance in the complex domain. In an embodiment, sensor 30 is a temperature sensor such as a thermocouple well known and widely used in the art. In various embodiments, both of sensors 28, 30 are the same type of sensor, i.e., sensors 28 and 30 are both ohmmeters or both temperature sensors. In further alternative embodiments, more than two sensors 28, 30 are included in ablation system 22. In one such embodiment, one ohmmeter and two thermocouples are components of ablation system 22.

Both sensors 28, 30 and at least one of ablation member 26 and source of ablation energy 32 are coupled to controller 36. In an embodiment, source of ablation energy 32 is coupled to controller 36. Controller 36 includes electronic componentry well known in the art for receiving and processing data received from sensors 28, 30 and controlling the output from ablation member 26 and source of ablation energy 32. In various embodiments, controller 36 is additionally coupled to user interface 38, by which controller 36 in particular and ablation system 22 in general may be controlled, at least in part, by a user. In various embodiments, controller 36 is further coupled to input 40 for receiving programming instructions and other computing data.

Head 24 may further incorporate vacuum source 42 connected to vacuum ports 44 in head 24 by way of a conduit 45 in neck 34 (obscured). When head 24 is placed against tissue 11 of heart 10 a zone of low pressure may be created between head 24 and heart 10, which may tend to secure, at least in part, head 24 against heart 10. This may bring ablation member 26 into adequate proximity of heart 10 to ablate tissue 11, and it may bring sensors 28, 30 into adequate contact with heart 10 to detect characteristics such as impedance and temperature of proximate tissue 11 of heart 10.

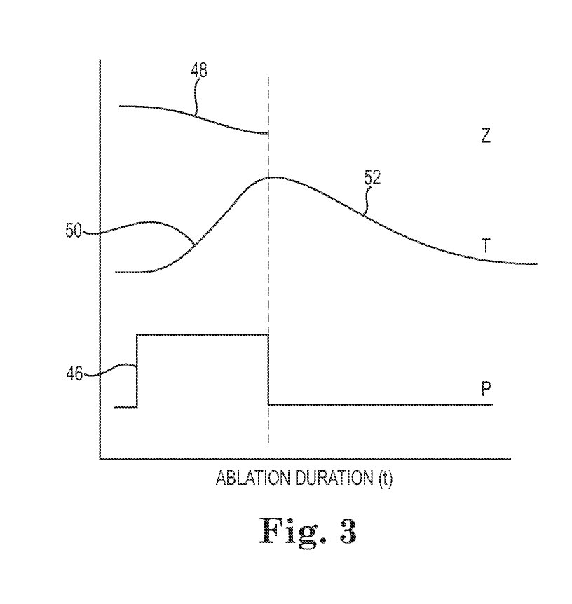

FIG. 3 is a graphical diagram depicting a sensed response in tissue 11 to a test pulse of ablation energy administered by ablation member 26. In an embodiment, after head 24 has been positioned with respect to tissue 11, source of ablation energy 32 delivers low amplitude pulse 46 of ablation energy to tissue 11. Impedance sensor 28 senses impedance response 48 in tissue 11, while temperature sensor 30 senses temperature response 50 in tissue 11. Test pulse 46 may be of various lengths, from a fraction of a second to a minute or more, and may be anywhere up to one hundred watts or more, dependant on circumstances. In various embodiments, test pulse 46 lasts for between ten seconds and twenty seconds and has a power of between ten watts and sixty watts. In an embodiment, test pulse 46 is forty watts for fifteen seconds.

When test pulse 46 is applied to tissue 11, the impedance of tissue 11 and cardiac tissue proximate tissue 11 may tend to decline over time during the period of test pulse 46. For example, the impedance of tissue 11 may tend to decay according to response 48, in which an initial gradual decay is followed by a period of rapid decay followed by a second period of gradual decay. In various embodiments, the second period of gradual decay occurs as the impedance of tissue 11 approaches a lower limit.

In certain circumstances, when test pulse 46 turns off, impedance measurements may tend to become immediately unavailable. As such, in various embodiments, impedance measurements are only taken during the pendency of test pulse 46. However, impedance response curve 48 may be measured after the pendency of test pulse 46 when a valid curve is detectable due to latent propagation of electrical signals by cardiac tissue 11.

When test pulse 46 is applied to tissue 11, the temperature of tissue 11 and cardiac tissue proximate tissue 11 may tend to increase according to temperature response curve 50. After the pendency of test pulse 46, the temperature may tend to decrease according to post-pulse temperature response curve 52. As such, in various embodiments, temperature response curve 50 is measured both during and after the pendency of test pulse 46.

In various embodiments, test pulse 46 is delivered once and at least one of impedance response curve 48 and temperature response curve 50 is measured. In an embodiment, both are measured during the pendency of test pulse 46, and temperature response curve 52 is measured after the pendency of test pulse 46. In an alternative embodiment, impedance response curve 48 is measured during test pulse 46 while temperature response curve 52 is measured after test pulse 46.

In further alternative embodiments, two test pulses 46 are delivered. In such an embodiment, one of impedance response curve 48 and temperature response curve 50 is measured during the first of test pulses 46, while the other is measured during the second of the test pulses 46. In an embodiment, temperature response curve 50 is measured first, both during and after first test pulse 46. After temperature response curve 50 is measured, second test pulse 46 is delivered and impedance response curve 48 is measured.

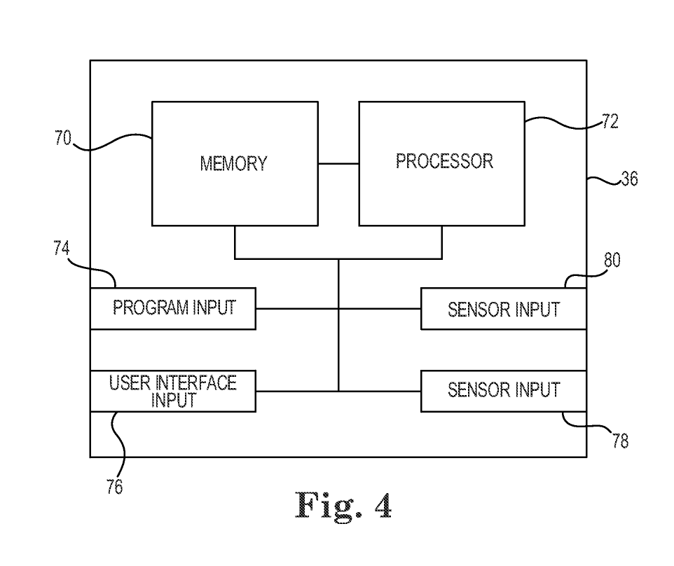

When test pulses have been sensed by sensors 28, 30, data indicative of curves 48, 50 may be transmitted from sensors 28, 30 to controller 36. FIG. 4 is a block diagram of an embodiment of controller 36. In various embodiments, controller 36 includes memory 70 and processor 72, as well as inputs 74, 76, 78, 80 from user interface 38, program input 40 and from sensors 28, 30, respectively. Memory 70 and processor 72 may be selected from any number of suitable commercially available components.

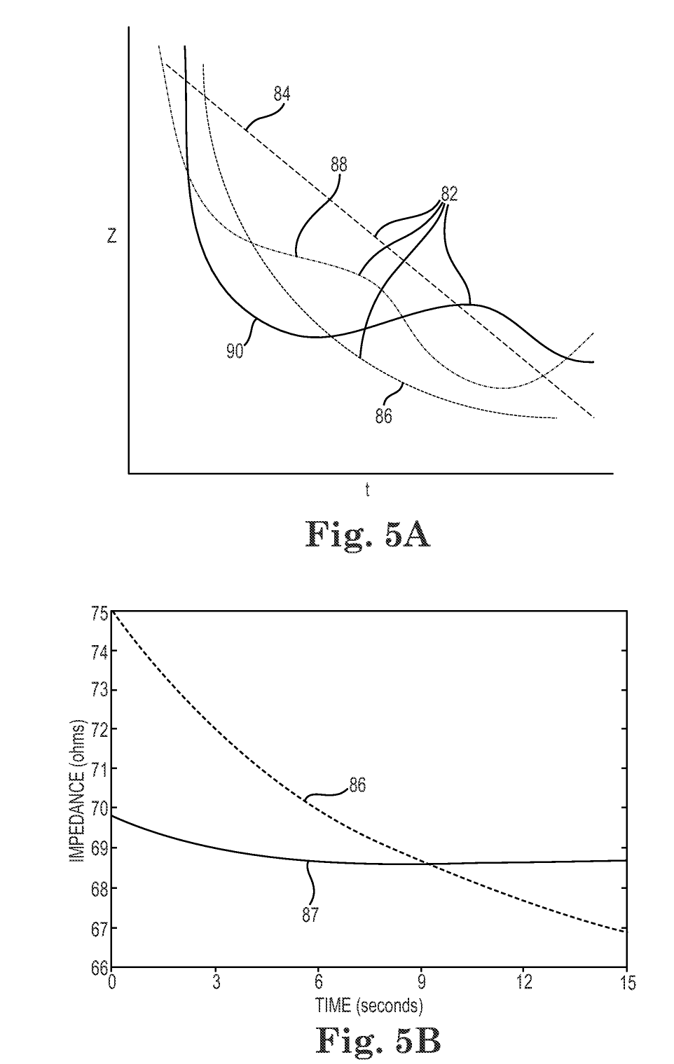

Memory 70 may be loaded by way of user interface 38 or program input 40 with predetermined response curves 82 for impedance and temperature (FIGS. 5A and 5B depict predetermined impedance response curves). In an embodiment, at least two response curves for each of impedance and temperature are loaded into memory 70. In alternative embodiments, at least six curves of each of impedance and temperature are loaded into memory 70. In further alternative embodiments, more than ten curves of each of impedance and temperature are loaded into memory 70.

Predetermined response curves 82 may, in an embodiment, be predetermined in a laboratory setting. Such predetermined response curves 82 may be obtained on the basis of various known variables. For instance, one predetermined response curve 86 may correspond with the impedance response of tissue to a particular ablation element 26 being utilized on tissue 6.3 millimeters thick and having a low blood flow, e.g., less than 2 L/minute, for fifteen seconds at forty Watts. A second predetermined impedance response 87 curve may be obtained with the same ablation element 26 being utilized on tissue 1.5 millimeters thick with a higher blood flow, e.g., greater than 4 L/minute, for fifteen seconds at forty Watts. Various additional combinations may be included with varying depths and blood flows. Length of test pulse 46 may also be varied.

In an embodiment, memory 70 is loaded with response curves which correspond to one ablation element 26. If ablation element 26 is replaceable or swappable, then new response curves corresponding to new ablation element 26 may be loaded into memory 70. Alternatively, response curves for multiple ablation elements 26 may be included for ablation systems 22 which include swappable or replaceable ablation elements 26. Additionally, further response curves may be developed for test pulses at varying power levels and time durations.

As shown, predetermined response curves 82 may be linear 84, quadratic 86, cubic 88, fourth degree 90, or logarithmic. Each may represent a particular response of test tissue to test pulse 46. Processor 72, by comparing response curve 48, 50 against the various predetermined response curves 82, determines a best-fit predetermined response curve 82 for a particular response curve 48, 50. The tissue characteristics, such as thickness and blood flow, which correspond to predetermined response curve 82 are, in an embodiment, thus taken as useful approximations of the characteristics of tissue 11.

In embodiments in which both impedance response curve 48 and temperature response curve 50 are obtained, both may be utilized in determining best-fit predetermined response curves 82. In various embodiments, one best-fit predetermined response curve 82 is obtained for each of response curve 48, 50. In an embodiment, the best-fit predetermined response curves 82 may then be combined as an aggregate best-fit response curve, which is then applied to determine useful approximations of the characteristics of tissue 11. In an alternative embodiment, each best-fit predetermined response curve 82 is utilized to obtain approximations of characteristics of tissue 11, and then the approximations are aggregated to obtain an aggregate approximation of characteristics of tissue 11, which may then be utilized in delivering therapy.

In further alternative embodiments, response curves 48, 50 may themselves be aggregated and applied to determine a single best-fit predetermined response curve 82. In an embodiment, response curves 48, 50 may be aggregated as multi-order polynomials. In alternative embodiments, response curves 48, 50 may be aggregated as multi-dimensional curves. In such an embodiment, predetermined response curves 82 may be multi-dimensional as well.

In various embodiments, an automated best-fit algorithm is utilized by processor 72 to determine the best-fit predetermined curve 82 for a particular response curve 48 or combination of response curves 48, 50. In an embodiment, the best-fit predetermined response curve 82 is determined according to a common commercially available algorithm, such as is conducted by MathWorks MATLAB™ program from The Mathworks, Inc. In alternative embodiments, relatively simpler algorithms are applied. In an embodiment, change per unit time between response curve 48 and predetermined response curves 82 is compared. In an alternative embodiment, the average derivative of the curve over a set period of time in response curve 48 and in predetermined response curves 82 are compared. In an alternative embodiment, the percentage change per unit time between response curve 48 and predetermined response curves 82 is compared. In various alternative embodiments, some of these methods are utilized in combination. In an embodiment, all of these methods are utilized in combination. In an embodiment, the best-fit predetermined response curve 82 is selected by choosing the predetermined response curve 82 with the most methods closest to response curve 48.

In an alternative embodiment, best-fit predetermined response curve 82 may be selected, at least in part, on the basis of a user input. In an embodiment, controller 36 presents a graphical representation of response curve 48, 50 and predetermined response curves 82 to a user on user interface 38. By visually comparing response curve 48, 50 to predetermined response curves 82, a user may select a best-fit predetermined response curve 82 which will be applied to obtain approximations of characteristics of tissue 11. In various alternative embodiments, processor 72 may be utilized to determine a subset of predetermined response curves 82 to present to a user, and the user may make the final selection of best-fit predetermined response curve 82.

On the basis of the characteristics of the best-fit predetermined response curve 82, a full ablation procedure is selected by processor 72. For instance, if predetermined response curve 82 corresponds to tissue 2.5 millimeters thick and blood flow of more than 4 L/minute, an ablation procedure of a maximum of 72 Watts delivered for 1.5 minutes may be selected. If predetermined response curve 82 corresponds to tissue 3.0 millimeters thick and blood flow of less than 2 L/minute, an ablation procedure of a maximum of 65 Watts delivered for two minutes may be selected. On the basis of the ablation procedure selected, processor 72, or other componentry of controller 36, commands ablation member 26 or source of ablation energy 32 to deliver the ablation procedure to tissue 11 to form a lesion.

By pre-characterizing tissue 11, an ablation procedure may be conducted accurately without a need to take follow-up measurements to assess a condition of the forming lesion. Such an ability may save on componentry, complexity and cost of systems which do not need to incorporate further sensors and spend further time performing measurements. Alternative ablation procedures may be implemented which account for more and different factors taken both before and during ablation procedures. In various embodiments, the procedure may incorporate starting power P₀, and may have multiple additional selectable power levels. In various embodiments, the available selectable power levels may be any power level over a predetermined range consistent with the performance characteristics of ablation system 22. In an embodiment, the range is from thirty-five (35) watts to one hundred (100) watts, with selectable power levels variable within that range. In an embodiment, the range is continuous and all power values within the range are selectable. In alternative embodiments, the selectable power levels are discrete. In an embodiment, the selectable power levels include thirty-five (35) watts, sixty (60) watts, seventy (70) watts, eighty (80) watts, ninety (90) watts and one hundred (100) watts.

In addition to incorporating the initial impedance and temperature measurements, the procedure may incorporate ongoing inputs of parameters from sensors 28, 30, in various embodiments temperature and impedance. Based on the sensed parameters, controller 36 varies the ablation energy among the selectable power levels.

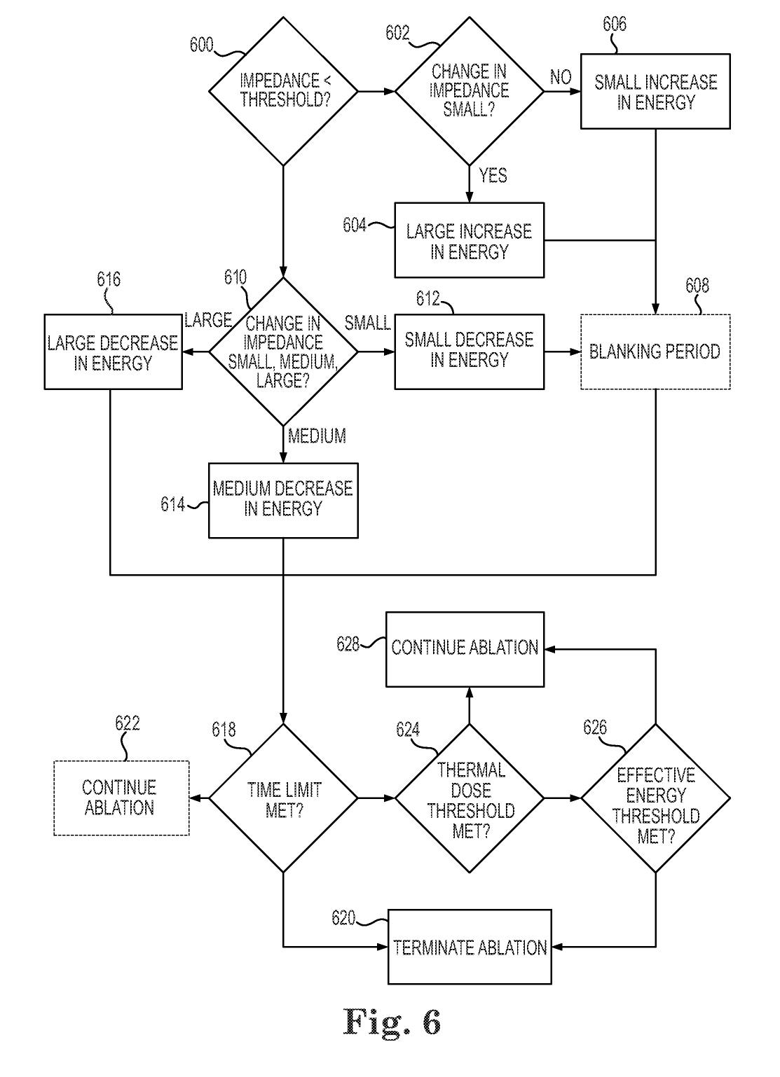

FIG. 6 is a flowchart for varying the delivered power during an ablation procedure. Such a procedure may advantageously be implemented after a pre-characterization of tissue 11, described above, in order to verify that a proper procedure has been selected and to make adjustments based on actual conditions following commencement of the procedure. Alternatively, power may be varied during an ablation procedure without regard to pre-characterizing tissue, which may save time in an operating room setting.

In various embodiments, a change in a sensed parameter over time may result in a change in the selected power. In an embodiment, if the first derivative of a measured impedance is less than a predetermined threshold for a predetermined period of time (600), a power plateau criteria may be met, suggesting a power level has been attained in which the change in sensed parameters indicate an increase in delivered power may be implemented. In various embodiments, the power plateau threshold and the number of data points which must meet the threshold to indicate a power plateau may be determined experimentally, depending on ablation member 26 and ablation device 22 generally. In certain embodiments, the power plateau threshold is met if the derivative of the impedance over time is less than or equal to two (2.0) in at least three of an immediately preceding five sample points. In an embodiment, the power plateau threshold is met if the derivative of the impedance over time is less than or equal to 1.3 in at least four of an immediately preceding five sample points.

If a power plateau is indicated, delivered power may be increased based, at least in part, on a change in the impedance (602). In embodiments where delivered power may be selected along a continuous range, an increase may be selected according to various factors. For instance, where the change in impedance is relatively low, such as when the derivative is less than 0.5, a relatively larger increase in delivered (604) power may be selected. Where the change in impedance is relatively larger, such as when the derivative is less than 1.3, but greater than 0.5, the increase in delivered (606) power may be relatively smaller. In embodiments where the delivered power is selected from discrete values, meeting the power plateau threshold may result in a one-step increase in delivered power. As such, in an embodiment in which the discrete power selections include thirty-five (35) watts, fifty (50) watts, sixty (60) watts and seventy (70) watts, and the current delivered power is fifty (50) watts, meeting the power plateau criteria would result in increasing delivered power to sixty (60) watts. In alternative embodiments, more than one step increase may be selected, and varying numbers of steps may be selected dependent on the change in impedance during the power plateau.

A power plateau blanking period may be applied (608). In a power plateau blanking period, input from sensors 28, 30 may be “blanked”, such as by ignoring input from sensors 28, 30, or by inhibiting sensors 28, 30 from sensing altogether. A blanking period may, for instance, provide a temperature of tissue 11 to respond to increased or decreased energy delivery before a new judgment is made as to whether the changed energy level is resulting in appropriate results. In various embodiments, the power plateau blanking period may be selectable based on patient conditions. In various embodiments, the power plateau blanking period is four (4) seconds or less. In an embodiment, the power plateau blanking period is 1.8 seconds.

In various embodiments, if various criteria are met, power delivery may be reduced (610). In various embodiments, the delivered power may be adjusted by variable amounts dependent on the amount of change in the measured impedance. In an embodiment, the relative change in delivered power may correspond to the relative change in impedance. In embodiments where the range of deliverable power is continuous, selected power may be adjusted to a fine resolution based on a change in impedance.

In embodiments where the values of deliverable power are discrete, decreases in power of various discrete steps among the selectable values may be applied dependant on the change in impedance, on the basis of a change in temperature, or both. For instance, if the current delivered power is eighty (80) watts, and the available steps are thirty-five (35) watts, fifty (50) watts, sixty (60) watts and seventy (70) watts, then a one-step drop would be to select seventy (70) watts, a two-step drop would be to select sixty (60) watts, and so forth. In various embodiments, if the change in impedance is relatively small, a one-step drop in delivered power may be implemented (612), if the change in impedance is relatively large, a three-step drop in delivered power may be implemented (616), and if the change in impedance is a medium change in impedance, a two-step drop in delivered power may be implemented (614). In an embodiment, a change in impedance is relatively small if the derivative of the impedance over time is greater than 1.3 for at least three of an immediately preceding five sample points, a change in impedance is medium if the change in impedance is greater than 3.0 at least two of an immediately preceding four sample points, and a change in impedance is relatively large if the change in impedance is greater than 5.5 at any time. Alternative values for what constitutes small, medium and large changes in impedance may be utilized in different circumstances. In addition, in alternative embodiments, more than three gradations may be applied. In an embodiment, five gradations are utilized.

In various embodiments, a post-step blanking period may be implemented (608) after a one-step decrease in delivered energy. In such embodiments, the post-decrease blanking period may be identical to the power plateau blanking period. In alternative embodiments, the post-decrease blanking period may be different from the power plateau blanking period. In some of the alternative embodiments, the post-decrease blanking period may be less than four seconds.

An ablation procedure may be terminated, i.e., the delivery of ablation energy is discontinued, according to various termination criteria or “thresholds.” In an embodiment, a time duration of the ablation procedure may be compared against a maximum allowable time limit (618). If the time limit is met, the ablation procedure is terminated (620). Optionally, if the time limit is not met the ablation procedure may be continued (622). In various embodiments, the maximum allowable time may vary according to a predetermined ablation procedure selected, as described above. In such embodiments, the predetermined time may depend on the thickness of tissue 11, the blood flow through and proximate tissue 11 and the nature of the energy delivery of the predetermined procedure itself. In various alternative embodiments, a fixed maximum time is provided. In one such embodiment, the fixed maximum time is one hundred twenty (120) seconds.

In various embodiments, alternative or additional termination criteria may be applied in addition to absolute time criteria. In an embodiment, when the absolute time limit is not met, ablation may be terminated (620) on the basis of a delivered thermal dose (624), i.e., the accumulated effective temperature as a function of time, e.g., degrees Celsius·seconds; and a delivered effective energy, i.e., an accumulated effective energy (626) over time. If both the thermal dose threshold and the effective energy threshold are not met, ablation may be continued (628). In alternative embodiments, ablation may be terminated on the basis of one of thermal dose and effective energy, but not the other.

When ablating tissue 11, certain effective temperatures may apply relating to the surface temperature of tissue 11 at which cell necrosis in tissue 11 starts to occur. For temperatures below the threshold effective temperature, cell necrosis may occur very slowly or not at all; for instance, it is the fact that cell necrosis does not occur at very low temperatures that allows tissue 11 to be pre-characterized prior to ablation, as described above. Above the threshold effective temperature, however, cell necrosis may occur comparatively rapidly, with increases in the rate of cell necrosis corresponding to some degree to the extent to which the surface temperature exceeds the threshold effective temperature.

In various embodiments, a thermal dose may be determined from the measured surface temperature of tissue 11 as a function of the number of times when the surface temperature exceeds the threshold effective temperature. The “measured surface temperature” is the temperature measured at the surface of the tissue by a sensor. The “effective temperature” is the temperature at which relatively rapid cell necrosis in the tissue occurs, e.g., a range of about 50 degrees Celsius to about 60 degrees Celsius. In certain embodiments the threshold effective temperature may be 55 degrees Celsius. In an embodiment, the measured surface temperature 11 is measured by sensor 30 on the Celsius scale. To the extent that measured surface temperature 11 exceeds the threshold effective temperature, in an embodiment fifty-five (55) degrees Celsius, the measured surface temperature in degrees Celsius is added to a surface temperature summation. As such, when the measured surface temperature is sixty (60) degrees Celsius, sixty is incorporated into the summation. When the measured surface temperature is fifty (50) degrees Celsius, nothing is incorporated into the summation. In various embodiments, the threshold effective temperature either represents a minimum requirement or a value which must be exceeded. When the summed measured surface temperature readings in excess of the temperature threshold exceed a thermal dose threshold, an adequate thermal dose may be deemed to have been transmitted to tissue 11 to cause sufficient cell necrosis to result in an adequate lesion. In certain embodiments, the “measured surface temperature” does not exceed the effective temperature. For example, the measured surface temperature is dependent upon the type of sensor employed, the placement of the sensor, the tolerance of the tissue, etc. For example, in certain embodiments ablative energy is delivered to tissue to achieve an effective temperature, i.e., necrosis in the tissue, whilst the measured surface temperature of the tissue is less than that of the effective temperature, e.g., the measured surface temperature is about forty (40) degrees Celsius. In this case, the “threshold effective temperature” may be set to a temperature less than that of the effective temperature to account for the difference (e.g., 40 degrees).

In an embodiment, surface temperature is measured five times per second. In embodiments in which multiple measurements are taken per second, the effective temperature may, in certain embodiments, be divided by the number of times per second at which the temperature is measured in order to obtain a measurement of thermal dose delivered over a one-second timeframe. As such, in an embodiment with five measurements per second, each measurement may be divided by five and added together to obtain a thermal dose per second measurement. Alternative timeframes are also envisioned. By providing a thermal dose measurement per unit time the measurement may be comparable between and among systems and timeframes which are not necessarily identical. In alternative embodiments, surface temperature may be measured more or less frequently as equipment and other limitations may allow. In alternative embodiments in which sensor 30 senses the surface temperature continuously or with adequate frequency to create a response curve of surface temperature values, thermal dose may be determined as the integral of the curve during the times in which the surface temperature exceeds the threshold effective temperature.

In various alternative embodiments, thermal dose may be conducted on temperature scales other than the Celsius scale, including the Fahrenheit scale and the Kelvin scale. In alternative embodiments, thermal dose may be determined on the basis of occurrences in which the surface temperature exceeds the threshold temperature; the thermal dose is deemed to be met when the number of occurrences exceeds an occurrence threshold, without regard to the extent to which the temperature threshold is exceeded. In further alternative embodiments, the summed temperature values are not the absolute temperature values but rather an extent to which the temperature value exceeds the threshold effective temperature. Thus, for instance, if the surface temperature is sixty (60) degrees Celsius against an effective temperature threshold of fifty-five (55) degrees Celsius then five (5) is incorporated into the summation. In alternative embodiments, the thermal dose is not necessarily the summation of the surface temperatures exceeding the thermal dose, but rather is a function of other mathematical operations, such as multiplication and aggregate averaging.

When the total effective temperature exceeds the thermal dose threshold an indication may be provided that the desired thermal dose has been reached. In an embodiment, the thermal dose is 1000 degree-seconds as summed from the temperature values which are in excess of the threshold effective temperature. In various alternative embodiments the thermal dose ranges from 800 to 4800 degree-seconds. In embodiments which utilize thermal dose and not effective energy to terminate delivery of ablation energy, ablation energy is terminated upon meeting the thermal dose threshold.

Effective energy or effective power may be computed in a manner similar to that of thermal dose, in that effective energy represents the delivery of an instantaneous amount of energy which is effective in the creation of cellular necrosis. Similarly with thermal dose, energy may be deemed “effective” if it is adequate to cause relatively rapid cellular necrosis in tissue 11. An effective energy threshold may be set at the level of energy delivery from ablation members 26 adequate to cause cellular necrosis through a middle of tissue 11, in contrast to thermal dose which is sensitive largely to the surface temperature of tissue 11.

In an embodiment, the effective energy threshold is approximately forty (40) Watts-second. Alternative effective energy thresholds may be utilized in alternative embodiments. Similarly with thermal dose, an effective energy delivered to tissue 11 may be measured on the basis of delivered energy which exceeds the effective energy threshold per unit time. Because delivered energy is created by source of ablation energy 32, the amount of ablation energy delivered may not need to be measured by a sensor but rather may simply be known. In such embodiments, effective energy may be determined by integrating a curve representing energy delivered over time during the times in which the energy delivered exceeds the effective energy threshold. Alternatively, the energy delivered may be “sampled” periodically. In an embodiment, delivered energy is summed five times per second to the extent that the energy exceeds the effective energy threshold. In alternative embodiments, energy “sampling” occurs at various alternative periods both more and less frequently than five times per second.

In various embodiments, the total effective energy threshold is 1200 Watts-second. In alternative embodiments, ranges from 800 to 4800 Watts-second may be applicable. In particular, where tissue 11 is relatively thin then a relatively smaller total effective energy may be useful in creating a lesion. When tissue 11 is relatively thick a relatively higher total effective energy may be useful in creating a lesion.

In embodiments in which both thermal dose and effective energy are measured, ablation is terminated when both the thermal dose and the effective energy thresholds are met. In an alternative embodiment, delivery of ablation energy is terminated when either of the thermal dose or effective energy thresholds are met. In various embodiments, only one of thermal dose and effective energy is considered, and delivery of ablation energy is terminated on the basis of meeting one of the thermal dose and effective energy requirements.

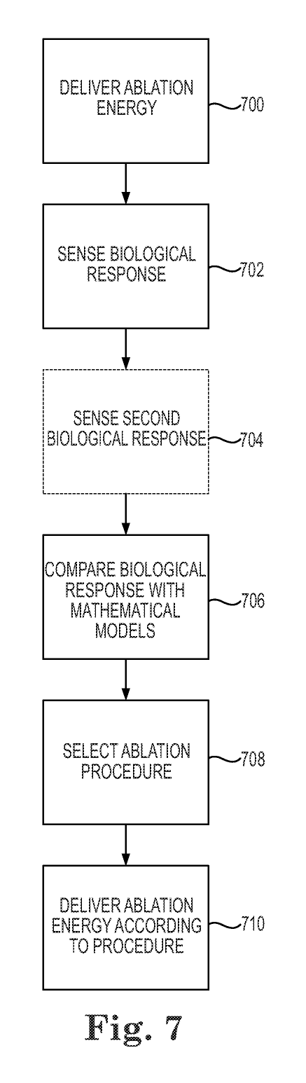

FIG. 7 is a flowchart of a method for ablating tissue. Ablation energy is delivered (700) to tissue 11 by way of ablation member 26. In an embodiment, the ablation energy is test pulse 46 of FIG. 3. A biological response is sensed by sensor 28 (702). In various disclosed embodiments, the biological response is impedance response 48 or temperature response 50. In an optional embodiment, a second biological response is also sensed (704). In such an embodiment, both impedance response 48 and temperature response 50 may be sensed. The biological response 48, 50 is compared (706) with a plurality of predetermined mathematical models 82, and an ablation procedure is selected (708) on the basis of the comparison. Ablation energy is delivered (710) to tissue 11 by way of ablation member 26 in accordance with the ablation procedure, as selected.

In various embodiments, delivering ablation energy (700) delivers first pulse of ablation energy 46. In various embodiments, sensing a biological response (702) delivers a second pulse of ablation energy. In some embodiments, the second pulse of ablation energy is smaller than first pulse 46. In an embodiment, the second pulse utilizes less energy than is needed to create a lesion in tissue 11.

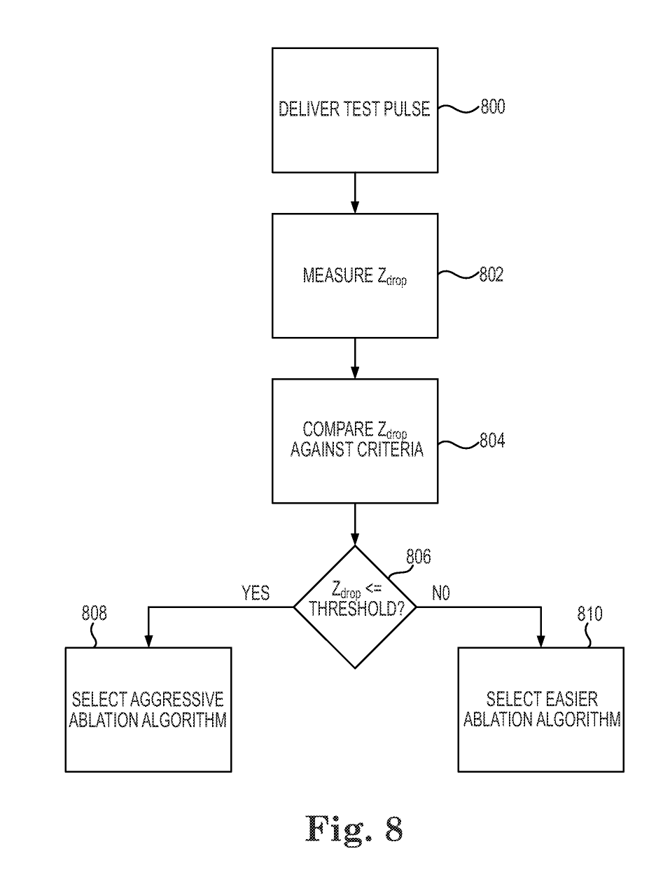

FIG. 8 is a flow chart of a particular embodiment of characterizing tissue consistent with the general flow chart shown in FIG. 7. A test pulse of ablation energy is delivered (800) to tissue 11 with a power of forty (40) watts for a duration of fifteen (15) seconds. The impedance drop of tissue 11 is measured (802) as a percentage according to the equation Z_{drop}=(Zₛₜₐᵣₜ−Zₘᵢₙ)/Zₛₜₐᵣₜ, where Zₛₜₐᵣₜ is the impedance of tissue 11 before or at commencement of delivery (800) of the test pulse, while Zₘᵢₙ is the minimum impedance of tissue 11 during the test pulse. Z_{drop} is then compared (804) against criteria for identifying tissue type. In various embodiments, if Z_{drop} is less than or equal to a threshold the impedance drop is small, while if Z_{drop} is greater than the threshold the impedance drop is large. In various embodiments, the threshold is in the range from three (3) percent to twenty (20) percent. In an embodiment, the threshold is seven (7) percent.

Where Z_{drop} is less than or equal to the threshold (806), tissue 11 is identified as difficult to heat. In various circumstances such a condition may be due to tissue 11 being relatively thin, because of relatively high blood or fluid flow, various alternative factors, or some combination thereof. A relatively aggressive ablation algorithm is selected (808) based on tissue 11 being difficult to heat. If Z_{drop} is greater than the threshold then a relatively weaker ablation algorithm is selected (810) based on tissue 11 being relatively easier to heat.

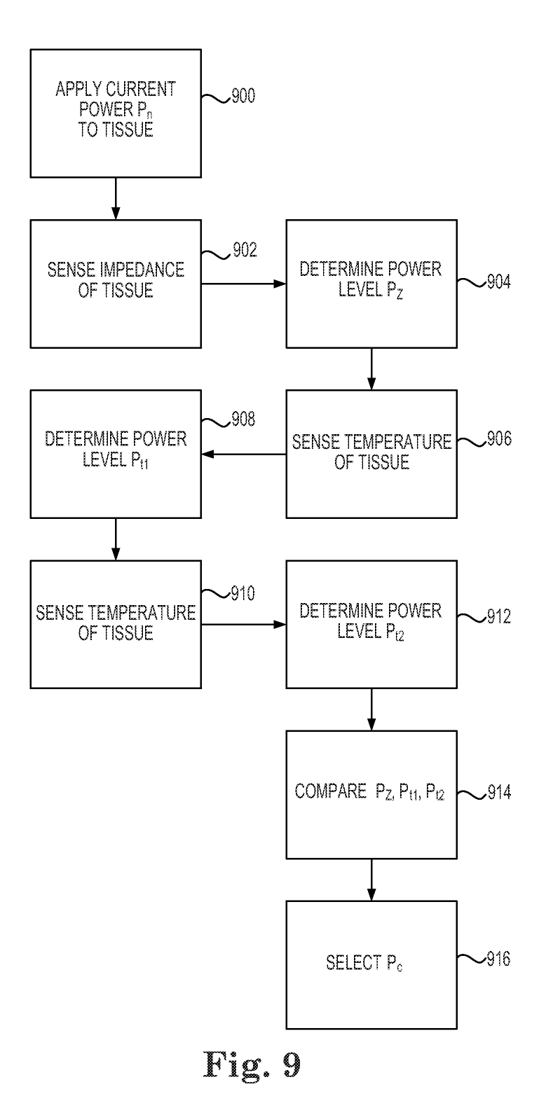

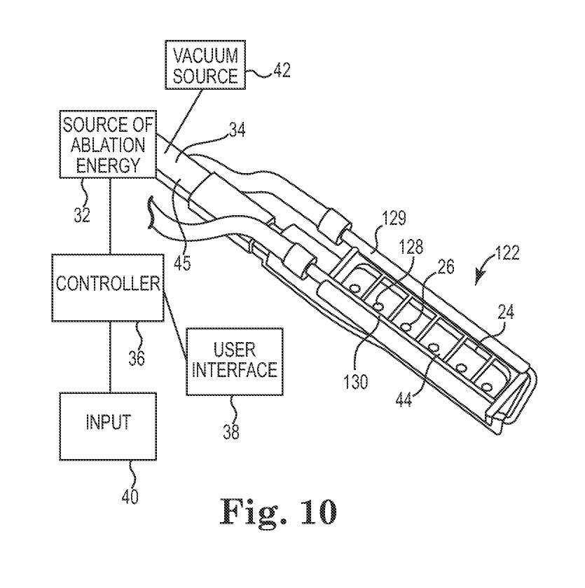

FIG. 9 is a flowchart for managing power modulation. A current power Pₙ is applied (900) to tissue 11. Various responses of tissue 11 to power Pₙ are measured. Ablation system 122 (FIG. 10), incorporating head 24 similar in most respects to that of ablation system 22 (FIG. 2) and utilized in FIG. 9 incorporates one ohmmeter 128 and two thermocouples 129, 130. It is noted that the flowchart of FIG. 9 may be modified to incorporate ablation systems with more or fewer sensors 28, 30 in ways which will be apparent to one skilled in the art. Ohmmeter 128 senses (902) an impedance of tissue 11 which provides the basis for controller 36 to determine (904) a power level P_{Z} at which ablation system 122 may deliver ablation energy to tissue 11. Thermocouple 129 senses (906) a temperature of tissue 11 at a first location which provides the basis for controller 36 to determine (908) a power level Pₜ₁ at which ablation system 122 may deliver ablation energy to tissue 11. Thermocouple 130 senses (910) a temperature of tissue 11 at a second location which provides the basis for controller 36 to determine (912) a power level Pₜ₂ at which ablation system 122 may deliver ablation energy to tissue 11.

As illustrated, components such as ablation elements 26, source of ablation energy 32, neck 34, user interface 38, input 40, vacuum source 42, vacuum ports 44 and conduit 45 are the same or essentially the same as those utilized in ablation system 22. In various embodiments, controller 36 determines each power level P_{Z}, Pₜ₁ and Pₜ₂ according to predetermined response curves 82 for initial values (FIG. 7), or according to starting power level P₀ and measured temperature and impedance (FIG. 6), depending on whether the controller is initializing ablation or delivering ablation. Once P_{Z}, Pₜ₁ and Pₜ₂ have been determined, P_{Z}, Pₜ₁ and Pₜ₂ are compared (914) and the minimum one selected (916) as P_{c}.

What is claimed is:

1. A method for ablating tissue, comprising:
delivering ablation energy, in one instance, to said tissue;
sensing a biological response in said tissue to said ablation energy; then
comparing said biological response with a plurality of predetermined mathematical models of predetermined biological responses of tissue to energy;
selecting one of a plurality of ablation procedures based on a result from said comparing step; and
delivering ablation energy, in another instance, to said tissue in accordance with a selected one of said plurality of ablation procedures.

2. The method of claim 1 wherein said ablation energy delivered in another instance creates a lesion in said tissue.

3. The method of claim 1 wherein said sensing a biological response step occurs after said delivering ablation energy, in one instance, step.

4. The method of claim 3 wherein said delivering ablation energy, in one instance, step delivers a first pulse of ablation energy, and wherein said sensing a biological response step comprises delivering a second pulse of ablation energy smaller than said first pulse.

5. The method of claim 4 wherein said second pulse of energy is less than an amount of energy necessary to ablate said tissue.

6. The method of claim 4 wherein said sensing a biological response step comprises sensing an impedance of said tissue.

7. The method of claim 1 wherein said sensing a biological response step occurs, at least in part, concurrently with said delivering ablation energy step.

8. The method of claim 7 wherein said biological response is a first biological response and further comprising the step, after said sensing a first biological response step, of sensing a second biological response in said tissue.

9. The method of claim 8 wherein said first biological response comprises an impedance of said tissue and said second biological response comprises a temperature of said tissue.

10. The method of claim 8 wherein said first biological response comprises a temperature of said tissue and said second biological response comprises an impedance of said tissue.

11. The method of claim 1 wherein said sensing a biological response comprises sensing an impedance of said tissue.

12. The method of claim 11 wherein said impedance comprises a complex impedance.

13. The method of claim 1 wherein said sensing a biological response comprises sensing a temperature of said tissue.

14. The method of claim 1 wherein said selecting step selects said ablation procedure from a plurality of predetermined ablation procedures.

15. The method of claim 14 wherein said ablation procedure is selected from a low power procedure, a long-term procedure, a high power procedure, a short-term procedure, a temperature set point procedure, a unipolar energy procedure, a bipolar energy procedure, a rise time procedure, cryo-energy procedure, a RF energy procedure, or any combination thereof.

16. The method of claim 1 wherein said ablation procedure comprises a series of ablation pulses delivered in sequence for a predetermined time.

17. The method of claim 1 wherein said tissue comprises heart tissue.

18. The method of claim 17 wherein said biological response is a function of a thickness of a wall of said heart.

19. The method of claim 18 wherein said biological response is a first biological response and further comprising the step, after said sensing a first biological response step, of sensing a second biological response in said tissue.

20. The method of claim 19 wherein said second biological response is a function of flow of blood in said heart.

21. The method of claim 1 wherein each of said plurality of mathematical models comprises a polynomial mathematical model.

22. A system for ablating tissue of a patient, comprising:
a source of ablation energy;
an ablation member, operatively coupled to said source of ablation energy, adapted to provide ablation energy to said tissue;
a sensing module which senses a biological characteristic of said tissue to said ablation energy delivered to said tissue from said ablation member; and
a controller, operatively coupled to said source of energy and said sensing module, said controller:
controlling said source of energy to deliver said ablation energy, in one instance, to said tissue through said ablation member;
determining a biological response in said tissue based on said biological characteristic sensed by said sensing module;
comparing said biological response with a plurality of predetermined mathematical models of said biological response to energy to obtain a comparison;
selecting an ablation procedure based on said comparison; and
controlling said source of energy to deliver said ablation energy, in another instance, to said tissue through said ablation member based on a selected one of a plurality of ablation procedures.

23. The system of claim 22 wherein controller creates a lesion in said tissue with said ablation energy delivered in one instance.

24. The system of claim 22 wherein said biological response occurs after delivery of said ablation energy delivered in one instance.

25. The system of claim 24 wherein said ablation energy delivered in one instance is a first pulse and wherein said controller delivers a second pulse of energy smaller than said first pulse.

26. The system of claim 25 wherein said second pulse of energy is less than an amount of energy necessary to ablate said tissue.

27. The system of claim 25 wherein said biological response comprises an impedance of said tissue.

28. The system of claim 22 wherein said biological response is a first biological response and wherein said sensing module senses a second biological characteristic in said tissue and said controller determines a second biological response based on said second biological characteristic.

29. The system of claim 28 wherein said first biological response comprises an impedance of said tissue and said second biological response comprises a temperature of said tissue.

30. A method of ablating tissue of a heart of a patient using an ablation device, comprising the steps of:
delivering ablation energy at an energy level value to said tissue of said patient with said ablation device;
determining a value of a temperature of said tissue and a value of an impedance of said tissue at a plurality of measurement times;
wherein said delivering ablation energy step is ceased at a time based, at least in part, on when at least one of:
an accumulated effective temperature of said tissue over time exceeds a predetermined thermal dose threshold, said accumulated effective temperature occurring when said value of temperature exceeds a temperature value at which any cell necrosis of said tissue occurs; and
an accumulated effective energy of said tissue over time exceeds a predetermined effective energy threshold, said effective energy occurring when said energy level exceeds a value of energy at which any cell necrosis occurs; and
if neither of said accumulated effective temperature exceeds said thermal dose threshold nor said accumulated effective energy exceeds said effective energy threshold, modifying said delivering ablation energy step by:
adjusting said energy level based, at least in part, on at least one of said temperature value being outside of a predetermined temperature range and said impedance value being outside of an predetermined impedance range; and
returning to said determining step.
